(19) United States
(12) Patent Application Publication
(10) Pub. No.: US 2019/0144893 A1
(43) Pub. Date: May 16, 2019

(54) A Method of Obtaining Useful Material from Plant Biomass Waste

(71) Applicant: Viridi Innovations Pty Ltd, Essendon (AU)

(72) Inventors: Enzo PALOMBO, Hawthorn (AU); David BEALE, Dutton Park (AU); Avinash KARPE, Hawthorn (AU); Ian H. HARDING, Hawthorn (AU)

(73) Assignee: Viridi Innovations Pty Ltd, Essendon (AU)

(21) Appl. No.: 16/098,608

(22) PCT Filed: May 2, 2017
(86) PCT No.: PCT/AU2017/050403

§ 371 Date: Nov. 2, 2018

(30) Foreign Application Priority Data
May 2, 2016 (AU) 2016901600

(51) Int. Cl. C12P 7/10; C12P 19/02; C12P 19/14

(52) U.S. Cl. CPC C12P 7/10; C12P 19/02; C12P 19/14; C12P 2201/00; C12Y 302/01008; C12Y 302/01021; C12Y 110/03002; C12Y 111/01014; C12Y 302/01004

(57) ABSTRACT

A method of obtaining useful material from plant biomass waste. The method uses sonication and/or microwave irradiation followed by sequential incubation with mixed fungal cultures. In particular, the method involves obtaining useful material from plant biomass waste comprising the steps of: a) subjecting the biomass waste to microwave irradiation and/or sonication; b) incubating the biomass waste from step a) with one or more enzymes extracted from Basidiomycete fungi; and c) incubating the biomass waste from step b) with one or more enzymes extracted from Ascomycete fungi.

### FIELD OF INVENTION

A method of obtaining useful material from plant biomass waste. The method uses sonication and/or microwave irradiation followed by sequential incubation with mixed fungal cultures.

### BACKGROUND OF THE INVENTION

Although the growth, harvesting and processing of crops is important in the supply and demand of foodstuffs around the world, a pressing concern to all farmers and agriculturists is the management of plant waste. Plant waste occurs in many forms, including stalks, stubble (stems), leaves and seed pods left in a field or orchard after harvest. Plant waste may also comprise other materials, such as husks, seeds, fibres or roots left over after a crop is processed into its commercial form. Plant waste sometimes represents more than half of the entire crop collected. The collection, storage, processing and disposal of plant waste are pressing issues.

Harvesting and processing sugarcane results in biomass waste which comprises bagasse, sugarcane tops, dry and green leaves. For each 10 tonnes of sugarcane crushed, approximately 3 tonnes of biomass waste is produced. When sugarcane biomass waste is left to rot, it breaks down and releases greenhouse gases, particularly methane which is 27 times more dangerous as a greenhouse gas than carbon dioxide, and it is also believed to have an impact upon ozone layer degradation. Furthermore wet cellulose, which is the principal component of sugarcane biomass waste ignites more easily than dry cellulose. This poses a problem for the safe storage of sugarcane biomass waste.

Grapes are a major global crop. Over 1.75 and 7.6 million tonnes of grapes were crushed for wine production during 2012 in Australia and the USA, respectively. Wine production produces large amounts of biomass waste. In 2013, global wine production of about 270 Megahecto-litres (Mhl) resulted in approximately 39 million tonnes of winery biomass waste.

Winery biomass waste consists of grape berries, plant-derived fibres, grape seeds, skin, marc, stalk and skin pulp. Winery biomass waste has limited use as animal feed stock due to poor nutrient value and low digestibility. This particular biomass waste also contains polyphenols which slow down decomposition and so, the majority of winery biomass waste ends up as toxic landfill. Hence, winery biomass waste has been classified as a pollutant by the European Union.

In the case of many grain crops over half of the material above-ground is not harvested. This is considered biomass waste. This biomass waste either takes the form of stubble, which consists of chaff, leaves and stalks, or straw which is the dried stalks of cereal plants such as wheat. Straw is nutritionally void and cannot be used for animal feed. Some farmers leave this biomass waste on the ground as mulch to prevent wind and water erosion, reduce evaporation, maintain soil carbon and to recycle nutrients. However, during heavy rainfall, the biomass waste can clog up machinery and harbour pests such as mice, slugs and weed seeds. Hence, many farmers choose to burn this type of biomass waste.

During the harvest of forest plantations, vegetation such as stem tops and branches are left behind in the landscape. This remaining vegetation is considered plantation biomass waste. With respect to the wood harvested and sent to mills, only half of the wood becomes a finished product. The remainder is waste made up of sawdust, woodchips, bark, planer and pole shavings. This for of material is referred to as sawmill biomass waste.

The degradation of biomass waste can generate useful industrial and medicinal biomolecules. Unfortunately, biomass has a complex structure which consists of cellulose and hemicellulose surrounded by lignin, which is very difficult to degrade. Various saprobic ascomycetes and saprobic basidiomycetes have been reported as effective biomass degraders. Enzymes such as cellulase and hemicellulase from the fungi also have the potential to be used to generate important molecules such as alcohols, flavonoids, organic acids and phenolics. However, depending on the fungi from which they originate, each of these enzymes has numerous limitations. One of which is their low comparative activity. Winery biomass degrading enzymes in particular suffer greatly from product inhibition, especially by cellobiose (direct inhibition) and glucose (indirect inhibition), even at low concentrations.

The degradation of biomass provides a useful source of important industrial and medicinal biomolecules. However, presently it is a process that is inefficient and subject to numerous limitations. There is therefore a need to develop a process for degrading plant biomass waste which is rapid and efficient in order to generate and access useful material.

### SUMMARY OF THE INVENTION

One of aspect of the present invention provides a method of obtaining useful material from plant biomass waste comprising the steps of:
a) subjecting the biomass waste to microwave irradiation and/or sonication;
b) incubating the biomass waste from step a) with one or more enzymes extracted from Basidiomycete fungi; and
c) incubating the biomass waste from step b) with one or more enzymes extracted from Ascomycete fungi.

In an embodiment, the biomass is subjected to microwave irradiation and sonication. In a further embodiment, the biomass is subjected to microwave irradiation for from about 1 minute to about 10 minutes. In a further embodiment, the biomass is subjected to microwave irradiation in an acidic environment. In a further embodiment, the biomass is subjected to sonication for from about 10 minutes to about 60 minutes. In a further embodiment, the biomass is subjected to sonication in a basic environment.

In an embodiment, one or more enzymes extracted from Basidiomycete fungi are extracted from at least one of Phanerochaete chrysosporium and Trametes versicolor. In a further embodiment, the biomass from step a) is incubated with a mixture of enzymes extracted from Phanerochaete chrysosporium and Trametes versicolor. In an embodiment, the one or more enzymes extracted from Ascomycete fungi are extracted from at least one of Aspergillus niger, Penicillium chrysogenum, Trichoderma harzianum and Penicillium citrinum. In a further embodiment, the biomass from step b) is incubated with a mixture of enzymes extracted from Aspergillus niger, Penicillium chrysogenum, Trichoderma harzianum and Penicillium citrinum. In a further embodiment, each incubation is for less than 24 hours.

In an embodiment, the plant biomass waste is comprised of sugarcane biomass waste, winery biomass waste, grain biomass waste, plantation biomass waste or sawmill biomass waste. In a further embodiment, the biomass is comprised of winery biomass waste.

### BRIEF DESCRIPTION OF THE DRAWINGS

FIG. 1—Analysis showing the lignin loss by grape biomass by sonication pre-treatment and fungal enzyme treatment at different concentrations of chemical used for sonication.

FIG. 2—Comparative analysis of reducing sugars in sonication pre-treated and enzyme degraded samples with control at different concentrations.

FIG. 3—Cellulase enzyme activity observed in sonicated and mixed enzyme degraded samples.

FIG. 4—β-glucosidase enzyme activity observed in sonicated and mixed enzyme degraded samples.

FIG. 5—Xylanase enzyme activity observed in sonicated and mixed enzyme degraded samples.

FIG. 6—Laccase enzyme activity observed in sonicated and mixed enzyme degraded samples.

FIG. 7—Lignin peroxidase enzyme activity observed in sonicated and mixed enzyme degraded samples.

FIG. 8—(A) PLS-DA score scatter plot of sonicated pre-treated mixed enzyme degraded grape biomass. R²X=99.2%, R²Y=98.5%, Q²=61.9%. (B) PLS-Da loading scatter plot of sonicated pre-treated mixed enzyme degraded grape biomass. R²X=99.2%, R²Y=98.5%, Q²=61.9%.

FIG. 9—Volcano Plot representing the most significant metabolites generated during the mixed enzyme degradation followed by sonication process. Yellow circles represents the metabolites with FC ≥2 and p-value ≤0.05.

FIG. 10—HPLC analysis of crystals obtained by freeze dried sonicated grape biomass samples.

### DETAILED DESCRIPTION OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or groups of integers or steps but not the exclusion of any other integer or step or group of integers or steps.

In one aspect the invention may provide an approach for combining microbial, chemical and physical processing to improve the effectiveness and efficiency of the degradation of plant biomass waste. A further aspect of the invention may provide an improved yield of commercially important compounds. A further aspect of the invention may provide a reduction in the time associated with the treatment and degradation of plant biomass waste. In a further aspect of the invention, there may be provided a reduction of mass after processing. In one aspect there may be provided a final output from the process that is more readily degraded as compost, has reduced toxic effluents and may eliminate or minimise the landfill requirement.

An aspect of the present invention is to obtain useful material from plant biomass waste. Useful material may be any material of interest, which includes the filtrates from the various steps of the process or the final output which may be readily degraded as compost. It is preferred that the useful material obtained from the plant biomass waste is/are product/s of commercial value, such as products useful for industrial or medicinal purposes. Examples of useful materials include, but are not limited to, tartaric acid, gallic acid, oxalic acid, malic acid, succinic acid, lithocholic acid, glycolic acid, N-glycolylneuraminic acid, citric acid, lactic acid, terephthalic acid, N-acetylgalactosamine, 5-hydroxytryptophan, resveratrol, anthocyanins, anthocyanidins, ethanol, butanol, phenolic compounds, flavonoids, carotenoids, terpenoids, vitamins, steroids and pigments.

In an embodiment, the useful material obtained from plant biomass waste comprises tartaric acid. Tartaric acid plays an important role in the production of wine. Tartaric acid lowers the pH of fermenting to a level where many undesirable bacteria cannot live and acts as a preservative after fermentation. Tartaric acid is also important in the field of pharmaceuticals. For example, tartaric acid is used in the production of effervescent salts, in order to improve the taste of oral medication. Tartaric acid also has several applications for industrial use. The acid has served in the farming and metal industries as a chelating agent for complexing micronutrients in soil fertiliser and for cleaning metal surfaces.

A person skilled in the art would understand that biomass refers to plants or plant-based materials which are not used for food or feed. In the present context, the term “plant biomass waste” refers to biomass that is a by-product of agricultural processes. This term does not include plants or plant-based materials that have been specifically cultivated for use in the generation of bioenergy. In an embodiment of the present invention, plant biomass waste comprises sugarcane biomass waste, winery biomass waste, grain biomass waste, plantation biomass waste or sawmill biomass waste. In a preferred embodiment, plant biomass waste comprises winery biomass waste.

In an aspect of the present invention, plant biomass waste is subjected to sonication and/or microwave irradiation. It has been found that pre-treatments such as sonication and microwave cause the breakdown of the lignin structure in plant biomass waste.

Without wishing to be bound by theory, the inventors understand that microwave irradiation serves to hydrolyse complex sugars such as cellulose and hemicelluloses. In the presence of an acidic solution, the microwave process is able to degrade larger saccharides into smaller sugars such as glucose, fructose and galactose (hexoses) and xylose, mannose and rhamnose (pentoses). In an embodiment of the invention, plant biomass waste is subjected to microwave irradiation for from about 1 minute to about 10 minutes. In a further preferred embodiment, plant biomass waste is subjected to microwave irradiation for from about 5 to about 8 minutes. In an embodiment, the plant biomass waste is subjected to microwave irradiation such that the temperature of the biomass is maintained at from about 150° C. to about 170° C. during the microwave treatment. In a further embodiment of the invention, the plant biomass waste is in an acidic environment when subjected to microwave irradiation. In a further embodiment, the acidic environment is a solution of about 1% H₂SO₄ to about 5% H₂SO₄. In a further embodiment of the invention, plant biomass waste is placed into an acidic environment and is subjected to microwave irradiation from about 1 minute to about 10 minutes. In a further embodiment of the invention, plant biomass waste is placed into an acidic environment and is subjected to microwave irradiation from about 5 minutes to about 8 minutes. In a further embodiment of the invention, plant biomass waste is placed into an acidic environment and is subjected to microwave irradiation from about 5 minutes to about 8 minutes and maintained at a temperature of from about 150° C. to about 170° C. during microwave treatment.

In one aspect, the filtered liquid resulting from the microwave process is pH neutralised and clarified using activated charcoal and an alkaline solution. The clarification process is understood to remove most of the inhibitors from the filtered liquid and increase the pH from very acidic to mildly acidic.

Sonication disrupts lignin which makes plant biomass degradation more effective. In an embodiment of the invention, the plant biomass waste is subjected to sonication for from about 10 minutes to about 60 minutes. In a preferred embodiment, the plant biomass is subjected to sonication for about 20 minutes. In another preferred embodiment, the plant biomass is subjected to sonication for about 40 minutes. In an embodiment of the invention, the plant biomass is in a basic environment when subjected to sonication. In a preferred embodiment, the basic environment comprises NaOH, KOH, MgOH or Ca(OH)₂. In a preferred embodiment, the basic environment is a solution which has about 0.25, 0.5, 0.75, 1, 1.25 or 1.5 molar concentrations of the aforementioned alkalis. In a preferred embodiment, the plant biomass waste is added to a solution of 1 M NaOH and subjected to sonication for 40 minutes. In a preferred embodiment, the plant biomass waste is added to a solution of 1 M NaOH and subjected to sonication for 20 minutes. In a preferred embodiment, plant biomass waste was added to a solution of 0.5 M KOH and subjected to sonication for 40 minutes. In a preferred embodiment, plant biomass waste was added to a solution of 0.5 M KOH and subjected to sonication for 20 minutes.

Various fungi such as Trichoderma sp., Aspergillus sp. and Penicillium sp. have been reported as biomass degraders owing to their ability to generate an array of enzymes such as endo- and exo-glucanases, β-glucosidase, xylanases, arabinofuranosidases and pectinases. This degradation generates useful industrial and medicinal biomolecules such as ethanol, flavonoids, phenolic compounds, anthocyanins and hydroxybenzoic acid. Additionally, fungi such as Penicillium spp. can be used for lignin mineralization during the degradation process. These fungi, and ultimately the enzymes derived from them, convert the lignocellulose complex to various soluble sugars, which can then be converted into other secondary products. However, due to the relatively recalcitrant nature of the lignocellulose complex, treatment by these fungi alone is still inefficient and/or ineffective.

An aspect of the present invention provides the use of mixed fungal cultures which results in a high production of degradative enzymes. Pre-treatments such as sonication and microwave combined with mixed fungal degradation can decrease biomass recalcitrance for more efficient breakdown, overcoming normal limitations. Combining said pre-treatments with mixed fungal degradation can produce up to 39 kg m⁻³ of reducing sugars and mineralise up to 18% of the lignin from plant biomass waste while reducing degradation time considerably.

In an aspect of the present invention, plant biomass waste is incubated with one or more enzymes extracted from Basidiomycete fungi. In an embodiment, one or more enzymes are extracted from Phanerochaete chrysosporium and/or Trametes versicolor. In an embodiment, the enzymes are extracted from Phanerochaete chrysosporium and Trametes versicolor. In an embodiment, enzymes from Phanerochaete chrysosporium and Trametes versicolor are added to the plant biomass waste in a 1:1 ratio. The incubation of the plant biomass waste with enzymes extracted from Basidiomycete fungi may span 15 to 24 hours at a temperature of about 35° C., for example.

An aspect of the invention provides the sequential fungal degradation of plant biomass waste. Following the treatment of one or more enzymes extracted from Basidiomycete fungi, in one embodiment, the plant biomass waste is incubated with one or more enzymes extracted from Ascomycete fungi. The purpose of the sequential enzyme degradation is to further improve degradation of the plant biomass waste and to generate products of interest such as ethanol, butanol, phenolic compounds and/or flavonoids.

In one embodiment of the invention, one or more enzymes are extracted from Aspergillus niger, Penicillium chrysogenum, Trichoderma harzianum and Penicillium citrinum. Extraction can be performed by routine techniques such as using an appropriate solvent or buffer, centrifugation, maceration, use of mortar and pestle filtration and/or sonication. In a further embodiment, a mixture of enzymes extracted from Aspergillus niger, Penicillium chrysogenum, Trichoderma harzianum and Penicillium citrinum is incubated with plant biomass waste. In a further preferred embodiment, the enzyme mixture extracted from Aspergillus niger, Penicillium chrysogenum, Trichoderma harzianum and Penicillium citrinum is in a percent ratio of 60:14:4:2 respectively. The incubation of plant biomass waste with a mixture of Ascomycete fungi may span 15 to 24 hours at temperatures of about 45° C. to about 55° C., for example.

The following examples provide preferred embodiments of the present invention.

### EXAMPLES

### Example 1—Extraction and Analysis of Metabolites from Grape Biomass Materials

Grape biomass of Vitis vinifera var. Cabernet was acquired from the Australian Wine Research Institute (AWRI), Glen Osmond, South Australia, Australia. The grape biomass was dried at 50° C. overnight and then used for experiments. Fungal cultures of Trichoderma harzianum and Penicillium chrysogenum were acquired from Agpath Pty Ltd., Vervale, Victoria, Australia. Fungal cultures of Aspergillus niger, Penicillium citrinum were obtained from the culture collection of Swinburne University of Technology. Trametes versicolor and Phanerochaete chrysosporium were kindly supplied by the culture collection of Manufacturing Flagship, Commonwealth Scientific and Industrial Research Organization (CSIRO), Clayton, Victoria, Australia. All fungi were cultured on aseptic Sabouraud Dextrose medium composed of Sabouraud Dextrose powder (30 g/L) and Agar (15 g/L).

The American Association of Textile Chemists and Colourists (AATCC) mineral salt iron medium, consisting of NH₄NO₃ (3 g/L), KH₂PO₄ (2.5 g/L), K₂HPO₄ (2 g/L), MgSO₄.7H₂O (0.2 g/L) and FeSO₄.7H₂O (0.1 g/L) with pH set at 5±0.2 was used with grape biomass in a 250 mL conical flask. AATCC medium (20 mL) with 20 g grape biomass was taken in a flask. All fungi except Phanerochaete chrysosporium were inoculated in this medium and incubated at 30° C. on a shaker at 150 rpm for 5 days. Phanerochaete chrysosporium was incubated at 37° C. with 120 rpm due to its differential optimum growth conditions. The fungal enzymes were quantified at 1×10⁻⁷ spores/mL. Enzyme extraction from these flasks was performed using 30 mL sodium citrate buffer (pH 4.8). The filtered enzyme solution was then used for enzyme degradation.

### Treatment of Plant Biomass Waste

Sonication pre-treatment was applied to grape biomass using a sonicator (Model: Q700; Qsonica, LLC., CT, USA). Alkaline sonication treatments were applied using concentration gradients of NaOH (1 M) and KOH (0.25, 0.5 and 1 M). Grape biomass (5 g) was mixed in 100 mL of the aforementioned alkaline solutions and sonicated in a glass beaker for 20 and 40 minutes (sonication parameters: Amplitude=100%, Power=700 W and Frequency=20 kHz). During optimisation, 20 minute was more efficient than 40 minutes. Therefore, for all experimental purposes 20 minutes of sonication was applied. Sonicated samples were filtered using Whatman paper no 1 (12 μm pore size) and rinsed thoroughly with distilled water. The treated samples were then used in sequential microbial degradation experiments.

Pre-treated grape biomass was further degraded using extracted fungal enzymes. Samples of pre-treated biomass (1 g) were placed in individual tubes. Phanerochaete chrysosporium and Trametes versicolor enzyme extracts were added and incubated at 37° C. for 18 hours. An Ascomycete enzyme mixture (4 mL) in a percent ratio of 60:14:4:2 of Aspergillus niger, Penicillium chrysogenum, Trichoderma harzianum and Penicillium citrinum respectively was added and the grape biomass was further incubated at 50° C. for 18 hours.

### Analysis of Final Output

Lignin content was determined as Acid Soluble Lignin (ASL) and Acid Insoluble Lignin (AIL) using the National Renewable Energy Laboratory (NREL) method. The final output sample (0.1 g) was incubated in 1 mL of H₂SO₄ (72%) at 30° C. for 1 hour. The acid-hydrolysed sample was then diluted to 4% H₂SO₄ with the addition of distilled water. The mixture was autoclaved at 121° C. for 1 hour and then allowed to cool to room temperature. The supernatant was collected as the ASL fraction after filtration. Acid Soluble Lignin was determined by the absorbance of the supernatant at 320 nm using the equation given below.

$$\% ASL = \frac{ABS \times volume \times Df}{ɛ \times {W}_{S 1} \times pathlength} \times 100$$

### Where,

ABS=absorbance at 320 nm

volume=volume of total filtrate (30.35 mL)

ε=absorptivity of biomass at 320 nm (30 L/g/cm)

W_{S1}=oven dried weight of the sample

pathlength=pathlength of the cell (1 cm)

Df=dilution factor

The pellet remaining after removal of the supernatant was thoroughly rinsed with distilled water and dried at 105° C. for 4 hours. The dried samples were then weighed and recorded as Acid Insoluble Residue (AIR). The AIR was kept in a muffle furnace at 575° C. for 4 hours to determine total ash content. Acid Insoluble lignin was determined by the ratio of the difference between dry acid insoluble residue and ash to the original dry weight of the grape biomass as given below.

$$\% AIL = \frac{( {W}_{S 2} - {W}_{S 3} )}{{W}_{S 1}} \times 100$$

### Where,

W_{S1}=oven dried weight of the sample

W_{S2}=weight of AIR

W_{S3}=weight of ash

The total % lignin was calculated using the equation given below:

Total % Lignin=% AIL+% ASL

Determination of reducing sugar content was carried out using dinitrosalicylic acid (DNSA) assay. Degraded grape biomass filtrate sample (100 μL) was mixed with DNSA reagent (900 μL) and incubated in boiling water bath for 5 minutes. The mixtures were then cooled in an ice bath in order to stop the reaction. The absorbance was taken at 540 nm to determine the concentration of reducing sugars. A glucose gradient was used to derive the standard reducing sugar.

Cellulase assays were performed and measured in terms of Filter Paper Activity (FPA) by taking sodium citrate buffer (1 mL, 0.05 M, pH 4.8) in a tube. Diluted enzyme (0.5 mL) filtrate was added to this buffer, followed by 50 mg Whatman No. 1 filter paper (≈6 cm×1 cm). The mixture was vortexed for about 10 seconds followed by 1 hour incubation at 50° C. Dinitrosalicylic reagent (3 mL) was added immediately and the mixture was kept in a boiling water bath for 5 minutes. The reaction was terminated on an ice bath. Deionised water (20 mL) was added to this reaction and the mixture was inverted several times. The paper pulp was allowed to settle over 20-30 minutes. Absorbance was taken at 540 nm to measure the total reducing sugars generated for determination of cellulose activity. One International Unit (IU) of cellulase is defined as the amount of enzyme required to liberate 1 μmol glucose per minute under assay conditions.

β-glucosidase assay was performed using a mixture of sodium acetate buffer (1 mL, 0.1 M, pH 5), p-nitrophenyl-β-D-glucosidase (pNPG) (0.5 mL, 0.02 M) and diluted enzyme (0.5 mL) samples. The mixture was incubated at 50° C. for 5 minutes. Na₂CO₃ (2 mL, 0.2 M) solution was then added to stop the reaction. The optical density was measured at 400 nm to determine the β-glucosidase activity. One IU of β-glucosidase is defined as the amount of enzyme required to liberate 1 μmol of p-nitrophenol per minute under assay conditions.

The xylanase assay was performed using Highley's method (Highley, 1997). Birchwood Xylan (1%, 0.9 mL), sodium citrate buffer (0.1 mL, 0.05 M, pH 5) with diluted enzyme sample were mixed. This mixture was incubated at 50° C. for 5 minutes. 1.5 mL of DNSA was added, mixed and heated at 100° C. for 5 minutes. The mixture was cooled in an ice bath to terminate the reaction and then kept at room temperature. The optical density was measured at 540 nm to determine the xylanase activity. One IU of xylanase is defined as the amount of enzyme required to liberate 1 μmol xylose per minute under assay conditions.

The laccase assay was performed by adding potassium phosphate buffer (2.2 mL, 0.1 M, pH 6.5) to 0.5 mL of an appropriately diluted enzyme sample. This mixture was equilibrated at 37° C. for 5 minutes. Syringaldazine (0.3 mL, 0.216 mM in methanol) was added and mixed by inversion of cuvette. Increase in the absorbance at 530 nm was recorded for 10 minutes. Difference of absorbance (AA530 nm) was obtained using the maximum linear rate for sample and blank to determine the laccase activity. One IU of laccase activity was defined as the amount of enzyme catalysing the oxidation of 1 μmole syringaldazine to form quinone per minute at 30° C., pH 6.5 in a 3 mL reaction mixture.

Lignin peroxidase assay was performed using veratryl alcohol (0.3 mL, 0.02 M) with 0.84 mL of 0.2 M Na₂HPO₄-Citric acid buffer (0.84 mL, 0.2 M, pH 4). Hydrogen peroxide (0.3 mL, 0.004 M) was then added with 1.56 mL of diluted enzyme sample. Absorbance at 310 nm was recorded for 5 minutes. Difference of absorbance (ΔA₃₁₀ nm) was obtained using the maximum linear rate for sample and blank to determine the lignin peroxidase activity. One IU of lignin peroxidase is defined as the amount of enzyme required to liberate 1 μmol veratraldehyde per minute under assay condition.

Final output was further analysed by gas chromatography-mass spectrometry (GC-MS). Processed samples (120 mg wet weight, equivalent to 40±2 mg dry weight) were prepared. Briefly, a 1.0 mL aliquot of methanol (LC grade, ScharLab, Sentemanat, Spain) was added. This mixture was vortexed for about 30 seconds followed by centrifugation at 573 g at 4° C. for 15 minutes. Adonitol (10 μg/mL, HPLC grade, Sigma-Aldrich, Castle Hill, NSW, Australia) was added as an internal standard. 50 μL of supernatant was transferred to a fresh 1.5 mL vial and dried in an RVC 2-18 centrifugal evaporator at 40° C./210 g (Martin Christ Gefriertrocknungsanlagen GmbH; Osterode, Germany). All samples were stored at −800° C. for further analysis.

The stored samples were volatalised by derivatisation for analysis by GC-MS. Methoxymine HCl (40 μL, 2% w/v in pyridine) was added, followed by vortexing at 37° C. in a thermomixer (Model: Comfort; Eppendorf South Pacific Pty. Ltd., North Ryde, NSW, Australia) at 1400 rpm for 45 minutes. Silylation was performed by adding 70 μL of N,O-bis(trimethylsilyl)trifluoroacetamide (BSTFA) in 1% Trimethylchlorosilane (TMCS) to complete the derivatisation. The mixture was centrifuged at 15700 g for 5 minutes and the supernatant was transferred to GC-MS vials. Pre-derivatised ¹³C-Sorbitol [Kovats Retention Index=1918.76, m/z=620.00 (10 μg/mL, HPLC grade, Sigma-Aldrich, Castle Hill, NSW, Australia)] was added as the second internal standard in order to verify instrument stability over the run time.

The GC-MS was performed using an Agilent 7890B GC oven coupled with a 5977A MS detector (Agilent Technologies, Mulgrave, Victoria, Australia). The GC-MS system was fixed with a 30 m HP-5MS column, 0.25 mm ID and 0.25 μm film thickness. All injections were performed in a split mode with 1 μL volume; the oven was held at an early temperature of 70° C. for 2 minutes and then increased to 300° C. at 7.5° C./min; the final temperature was held for 5 minutes. The transfer line was held at 280° C. and the detector voltage at 1054 V. Mass spectra was acquired from 45 to 550 m/z, at an acquisition frequency of 4 spectra/second the MS detector was turned off until the additional derivatisation reagent eluted from the column. Data acquisition and spectral examination was achieved using Agilent MassHunter quantitative analysis program. Qualitative analysis of the compounds was carried out according to the Metabolomics Standard Initiative (MSI).

Chemometric and statistical examination were carried out using SIMCA 13, a chemometric software package (Umetrics AG, Umeå, Sweden), and MetaboAnalyst 2.0, an online statistical package (TMIC, Edmonton, Canada) (Xia et al., 2012). Peaks of chromatography were important where the Fold change (FC) was >2.0 and P-values were <0.05. It was expected and observed that each profile analysed would comprise a collection of putative metabolites of mixed concentrations generated during the degradation process. The data generated by mass spectral analysis were thus normalised with respect to internal standards (RSD=16.45%), where a magnitude of 1 FC referred to a concentration of 10 mg/L. Any FC values of less than 0.5 were considered as fungal-utilised metabolites.

An unsupervised statistical approach using principal component analysis (PCA) was undertaken on the data, with no clear separation being observed. To accommodate the outliers and enable differentiation between the groups based on metabolic pattern, a partial least square-discriminant analysis (PLS-DA) was employed. This is a supervised method used to analyse large datasets and has the ability to assess linear/polynomial correlation between variable matrices by lowering the dimensions of the predictive model, enabling easy discrimination between samples and the metabolite features that cause the discrimination.

### Results and Discussion

From the process described above, it was observed that sonication with 0.5M KOH resulted in a better biomass degradation when compared to other conditions. This was observed in the mineralisation of lignin with 0.5 M KOH, where a 20 minute sonication resulted in about 13% lignin loss (FIG. 1). Other alkaline gradients such as 1 M KOH, 1 M NaOH and 0.25 M KOH were used for sonication pre-treatment. It was observed that 1 M KOH gave 12.8% lignin loss, 1M NaOH gave 6.7% lignin loss and 0.25M KOH gave 2.8% lignin loss. After sonication pre-treatment, enzyme degradation resulted in a further degradation of 0.2% in 0.25 M KOH and 2.5% in 1 M NaOH samples. Negligible lignin content was degraded in 0.5 and 1 M KOH samples by enzyme degradation. Comparative analysis of sonication pre-treated and enzyme degraded with control is shown in FIG. 1.

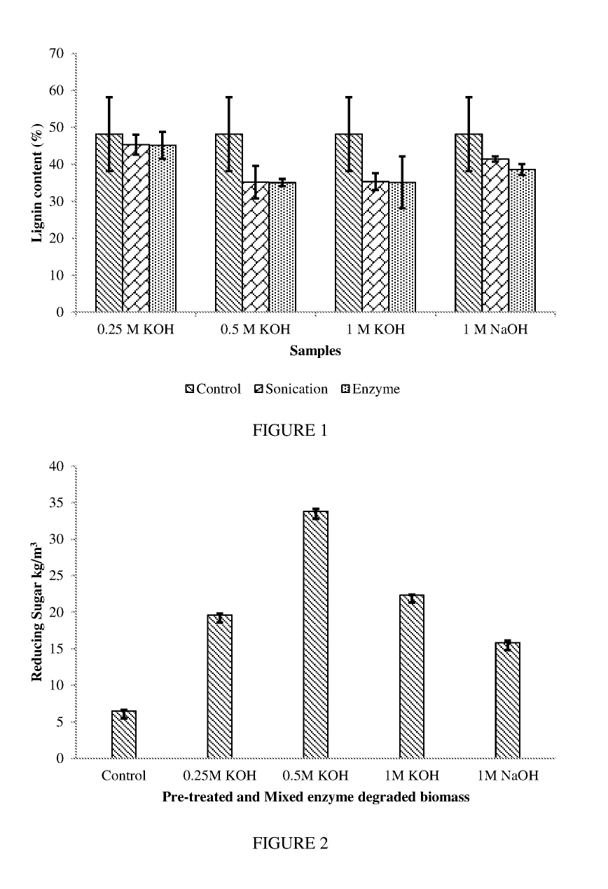

The highest amount of sugar yield was observed in the biomass subjected to sonication in 0.5 M KOH followed by enzyme treatment. The sugar yield observed in this sonicated sample with 0.5 M KOH sample was 33.8 kg/m³. This was followed by sonication with 1 M KOH, 0.25M KOH, 1M NaOH which resulted in 22.3, 19.6 and 15.8 kg/m³ sugars, respectively (FIG. 2).

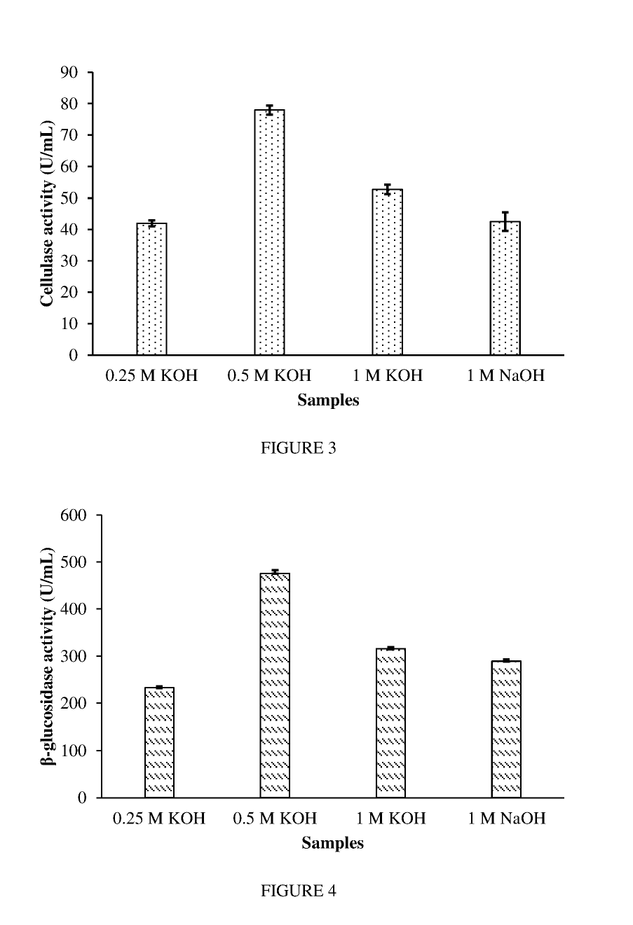

The highest cellulose degradation by enzyme mixture was observed in 0.5 M KOH assisted sonicated biomass at about 78 U/mL. This was followed by the samples sonicated after pre-treated with 1M KOH, 1M NaOH and 0.25M KOH. For these, the cellulase activities were observed at about 52.6, 42.5 and 42 U/mL, respectively (FIG. 3). Sonication pre-treatment by alkaline solution caused the lignin degradation. This resulted in the facilitation of cellulose and hemicellulose degradation by the enzymes, thereby causing an increase in cellulase activity.

The highest amount of β-glucosidase activity was observed in 0.5 M KOH assisted sonicated biomass at about 476 U/mL. Other alkaline treatments such as 1 M KOH, 1 M NaOH and 0.25 M KOH combined with sonication resulted in β-glucosidase activities of about 315.7, 289.4 and 233 U/mL, respectively. (FIG. 4). As the cellulase activity is seen to be high in 0.5 M KOH sample the β-glucosidase activity is also seen highest. This confirms that cellulase activity is not inhibited by β-glucosidase, cellobiose is efficiently converted to glucose.

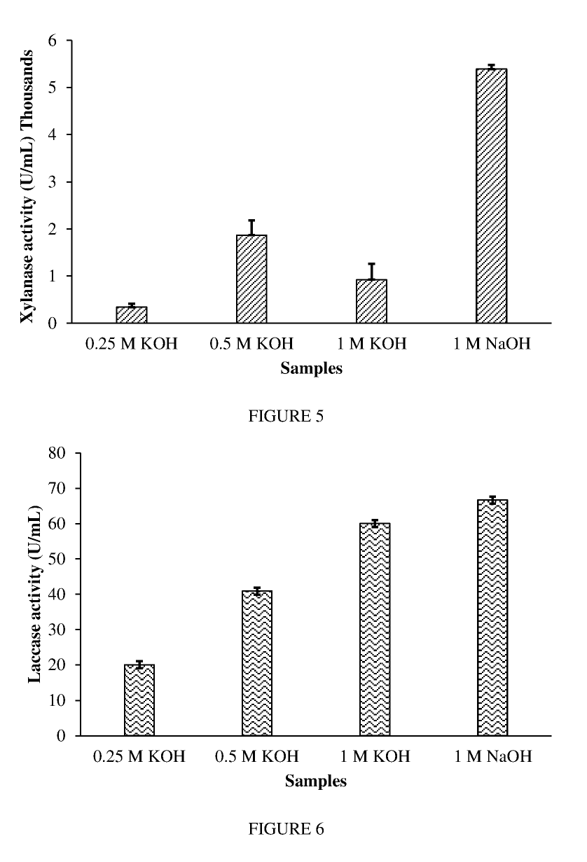

The highest amount of hemicellulose degradation was observed in 1 M NaOH assisted sonicated samples, with xylanase activities reaching about 5390.5 U/mL. This activity was considerably higher than in the biomass treated with other alkaline solutions. For example, 0.5 M KOH, 1 M KOH and 0.25 M KOH displayed xylanase activities of about 1867.3, 922.8 and 340.8 U/mL, respectively (FIG. 5). β-glucosidase and xylanase activities in 0.5 M KOH of 476 U/mL and 1867.3 U/mL respectively, were observed, which explain the enhanced breakdown of hemicelluloses and the highest observed lignin degradation.

The highest laccase activity was observed in 1 M NaOH assisted sonication at about 66.7 U/mL. Sonication treatment with other alkaline concentration gradients such as 1 M KOH, 0.5 M KOH and 0.25 M KOH resulted in laccase activities of about 60, 40.8 and 20 U/mL respectively (FIG. 6).

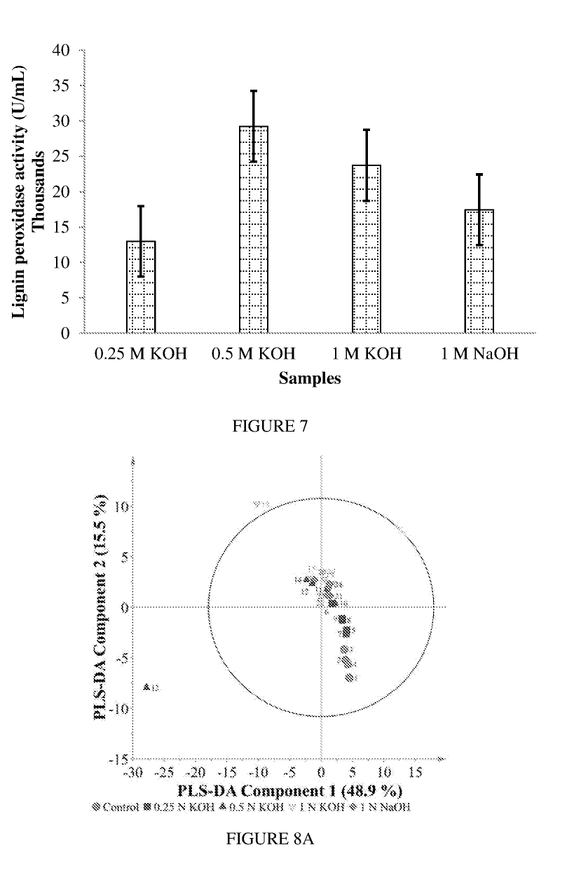

Considerably higher lignin peroxidase activities were observed during the experiments as compared to other enzymes under consideration. The highest lignin peroxidase activity of about 29231 U/mL was observed in 0.5 M KOH. Other alkaline solutions such as 1 M KOH, 1 M NaOH and 0.25 M KOH, combined with sonication, displayed lignin peroxidase activities of about 23730.7, 17461.5 and 12982.9 U/mL, respectively (FIG. 7).

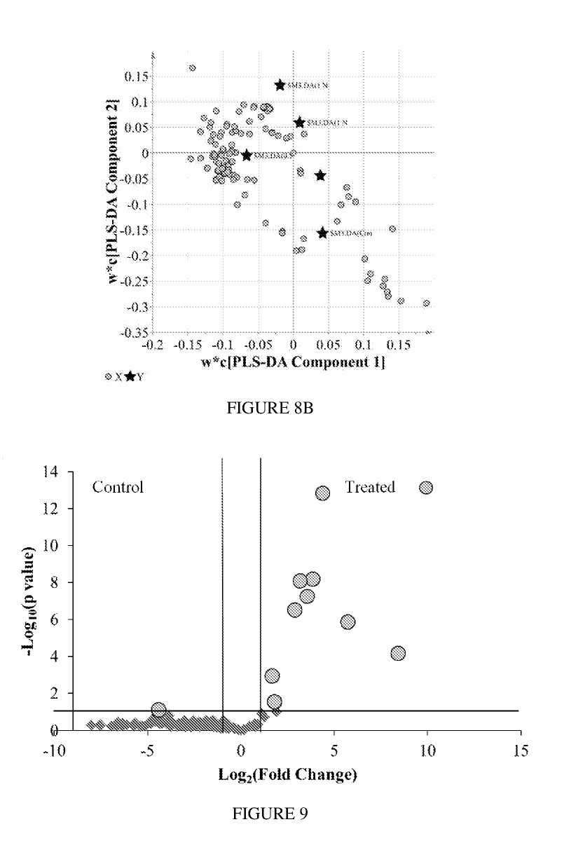

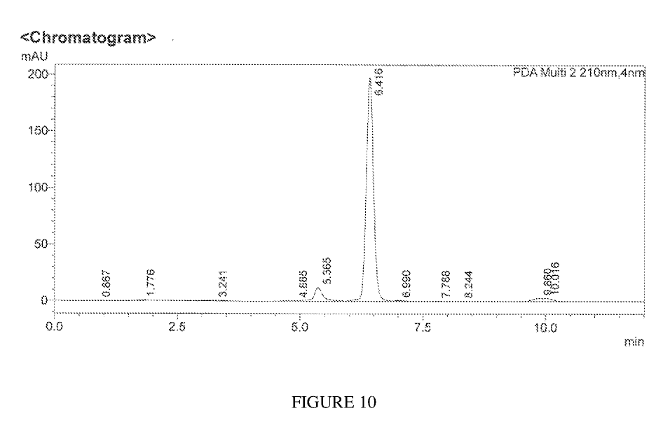

GC-MS analysis of treated and control samples indicated a presence of approximately 129 peaks, of which about 39 were considered as statistically significant (S/N ratio ≥50 with p-value ≤0.05). Univariate and multivariate statistical tools such as t-test, Principal component analysis (PCA) and Partial Least Square-Discriminant Analysis (PLS-DA) were used to analyse the distribution and classification of various metabolites. Due to the unsupervised nature, PCA was observed as a less satisfactory method to discriminate between the metabolite distributions (FIG. 8A). Due to this, samples were processed using Partial Least Square-Discriminant Analysis (PLS-DA) (FIG. 8B).

The volcano plot (FIG. 9) indicates the most significant metabolites. Fold Change (FC) value and P-values were used to classify the metabolites generated and consumed by the mixed enzyme degradation of grape biomass. Among 39 metabolites, 14 were observed to be generated in significant quantities (FIG. 9), while the remaining were consumed/metabolised during the biomass degradation process. The significantly generated metabolites included gallic acid, lithocholic acid, glycolic acid, citric acid, lactic acid. Other metabolites produced in considerable levels include octanoic acid, arabitol and succinic acid.

### Example 2—Tartaric Acid Production from Winery Biomass Waste Using Ultrasonication Treatment

### Materials and Methods

Grape biomass of Vitis vinifera var. Cabernet was acquired from the Australian Wine Research Institute (AWRI), Glen Osmond, SA, Australia. The grape biomass was dried at 50° C. overnight and was used for further analysis. Sonication pre-treatment was applied to the grape biomass using a sonicator (Model: Q700; Qsonica, LLC., CT, USA). Dried grape biomass (5 g) was mixed with 20 mL of distilled water and was sonicated for 10, 20 and 40 minutes. The sonication parameters used were: Amplitude=100%, Power=700 W and Frequency=20 kHz. During the optimization steps, 20 minutes of sonication were observed to be the most efficient. Therefore, for all experimental purposes, 20 minutes of sonication was applied. Sonicated samples were then centrifuged at 4000 g/15 minutes. The supernatant was transferred to a fresh tube and was frozen at −80° C. for 1 hour before drying. The frozen sample was freeze dried overnight (approximately 16 hours) to isolate the dissolved crystals. The dried crystals were analysed by GC-MS and HPLC in triplicate.

Crystal powder (40±2 mg dry weight) was mixed with 1.0 mL of methanol (LC grade, ScharLab, Sentemanat, Spain). This mixture was vortexed for about 30 seconds followed by centrifugation at 573 g at 4° C. for 15 minutes. ¹³C-Stearic acid (10 μg/mL, HPLC grade, Sigma-Aldrich, Castle Hill, NSW, Australia) was added as an internal standard. 50 μL of supernatant was then transferred to a fresh 1.5 mL vial and dried in an RVC 2-18 centrifugal evaporator at 40° C./210 g (Martin Christ Gefriertrocknungsanlagen GmnH; Osterode, Germany). All samples were then stored at −80° C. until further processing.

The samples were volatalised by dervatisation for application to GC-MS. Methoxymine HCl (40 μL, 2% w/v in pyridine) was added to the samples, followed by vortexing at 37° C. in a thermomixer (Model: Comfort; Eppendorf South Pacific Pty. Ltd., North Ryde, NSW, Australia) at 1400 rpm for 45 minutes. Silylation was performed by adding 70 μL of N,O-bis(trimethylsilyl)trifluoroacetamide (BSTFA) in 1% Trimethylchlorosilane (TMCS) to complete the derivatisation. The mixture was then centrifuged at 15700 g for 5 minutes and supernatant was transferred to GC-MS vials. Pre-derivatised ¹³C-Sorbitol [Kovats Retention Index=1918.76, m/z=620.00 (10 μg/mL, HPLC grade, Sigma-Aldrich, Castle Hill, NSW, Australia)] was added as the second internal standard at this point in order to verify instrument stability over the run time.

GC-MS was performed using Agilent 7890B GC oven coupled with a 5977A MS detector (Agilent Technologies, Mulgrave, Victoria, Australia). The GC-MS system was fixed with a 30 m HP-5MS column, 0.25 mm ID and 0.25 μm film thickness. All injections were done in a split mode with 1 μL volume; the oven was held at an early temperature of 70° C. for 2 minutes and then increased to 300° C. at 7.5° C./min; the final temperature was held for 5 minutes. The transfer line was held at 280° C. and the detector voltage at 1054 V. Mass spectra were acquired from 45 to 550 m/z, at an acquisition frequency of 4 spectra/second. The MS detector was turned off until the additional derivatisation reagent was eluted from the column. Data acquisition and spectral examination were achieved using the Agilent MassHunter quantitative analysis program. Qualitative analysis of the compounds was carried out according to the Metabolomics Standard Initiative (MSI).

The GC-MS process revealed a semi-quantitative output of composition of crystal samples. To quantify the amount and purity of organic acids produced by sonication, HPLC analysis was performed. The samples were dissolved in 20 mM sodium phosphate buffer (pH 2.5, 1 mg/mL). HPLC was performed using a Shimadzu LC-VP system with SCL 20A software, LC-20 ADVP pump, SIL-20 AVP autosampler, column oven (CT0 20 AVP) and SPD-M 20 AVP photo diode array detector. The separation was performed using a Grace-Prevail RP-18 column (Dimensions: 150 mm×4.6 mm ID, 5 μm pore size). The oven temperature was maintained at 30° C., whereas, the detector temperature was maintained at 40° C. Sodium phosphate (20 mM, pH 2.5) was used as the mobile phase in an isocratic condition. The flow rate was maintained at 0.5 mL/minute and absorbance of detector was kept at 210 nm for sample elution.

Chemometric and statistical examination were carried out using SIMCA 13, a chemometric software package (Umetrics AG, Umea, Sweden), and MetaboAnalyst 2.0, an online statistical package (TMIC, Edmonton, Canada) (Xia et al., 2012). Chromatography peaks were considered important where Fold Change (FC) was >2.0 and P-values were ≤0.05. The data generated by mass spectral analyses were thus normalised with respect to internal standards (RSD=16.45%), where a magnitude of 1 FC referred to a concentration of 10 mg L⁻¹. The data generated by HPLC were analysed by post-run analysis using Labsolutions platform (Shimadzu). Tartaric acid, malic acid, oxalic acid and succinic acid (0.1-5.0 g/L) were used as the calibration standards, against which the freeze dried crystals were analysed.

### Results

It was observed that the ultrasonication process at 100% amplitude increased the temperature of the system up to 90° C., causing water evaporation. This necessitated the use of temperature control during the entire process. Upon centrifugation, the slurry resulted in a colourless aqueous supernatant, indicating the absence of any phenolics, therefore, indicating no breakdown of lignin and minimal hydrolysis of cellulose/hemicellulose structures. Ultrasonication for 10 minutes resulted in a yield of 369 mg of colourless crystals (dry weight), while a 20 minute process yielded 558 mg (dry weight) from 5 g biomass.

GC-MS analysis showed the presence of oxalic acid, tartaric acid, malic acid and succinic acid, along with trace levels of glucose, fructose and galacturonic acid. Tartaric acid was observed as the major compound with the maximum peak area and, thus, the highest Fold Change value (FC=12.6) as normalized against the internal standards. Similar results were obtained by HPLC analysis, where tartaric acid showed the biggest peak area, followed by oxalic acid. It was observed that the largest composition of this mixture was tartaric acid (57.13%), followed by oxalic acid (3%), malic acid (0.71%) and succinic acid (0.24%). Therefore, the general yields for these acids (g/kg dried biomass) were 5.7%, 0.3%, 0.07% and 0.02%, respectively. GC-MS data and the previously reported data indicated that the sonication process at an 100% amplitude not only increased the temperature of the system beyond the required range (70° C.), but also causes a minimal, but observable breakdown of cellulose/hemicellulose structure, thereby releasing sugars, such as glucose and fructose, and uronic acids. These metabolites were thus observed as chemical contaminants in dried tartaric acid samples obtained. It was also possible that galacturonic acid was mistakenly observed as oxalic acid, owing to their similar retention times. It is therefore proposed that lower amplitude levels of ultrasonication (60-70%) at 60-70° C. will be able to yield higher levels of tartaric acid and minimize the contamination by sugars and uronic acids.

### Example 3—Industrial Scale Processing of Plant Biomass Waste

Grape biomass is pre-treated by microwave power (in 1% H₂SO₄) for various time periods (2-6 minutes). The liquid supernatant is removed and neutralised, followed by Saccharomyces cerevisiae yeast fermentation to generate ethanol. The remaining biomass is sonicated (in 2.8% KOH) for 20 minutes. The resultant filtrate is discarded and biomass further degraded by mixed fungal enzymes of Basidiomycetes (Ph. chrysosporium and T. versicolor in a percent ratio of 1:1) for 18-20 hours. Further degradation is achieved using Ascomycete enzymes (A. niger, P. chrysogenum, T. harzianum and P. citrinum in a percent ratio of 60:14:4:2) for 18-20 hours. The resultant metabolites produced during this fermentation is analysed by GC-MS.

### REFERENCES

HIGHLEY, T. L. 1997. Carbohydrolase assays. In: DASHEK, W. V. (ed.) Methods in plant biochemistry and molecular biology. CRC Press.
XIA, J., MANDAL, R., SINELNIKOV, I. V., BROADHURST, D. & WISHART, D. 2012. MetaboAnalyst 2.0—a comprehensive server for metabolomic data analysis. Nucleic Acids Res., 40, W127-W133.

1. A method of obtaining useful material from plant biomass waste comprising the steps of:
a) subjecting the biomass waste to microwave irradiation and/or sonication;
b) incubating the biomass waste from step a) with one or more enzymes extracted from Basidiomycete fungi; and
c) incubating the biomass waste from step b) with one or more enzymes extracted from Ascomycete fungi.

2. The method according to claim 1, wherein the biomass is subjected to microwave irradiation and sonication.

3. The method according to claim 1, wherein the biomass is subjected to microwave irradiation for from about 1 minute to about 10 minutes.

4. The method according to claim 1, wherein the biomass is subjected to microwave irradiation in an acidic environment.

5. The method according to claim 1, wherein the biomass is subjected to sonication for from about 10 minutes to about 60 minutes.

6. The method according to claim 1, wherein the biomass is subjected to sonication in a basic environment.

7. The method according to claim 1, wherein the one or more enzymes extracted from Basidiomycete fungi are extracted from at least one of Ph. chrysosporium and T. versicolor.

8. The method according to claim 7, wherein the biomass from step a) is incubated with a mixture of enzymes extracted from Ph. chrysosporium and T. versicolor.

9. The method according to claim 1, wherein the one or more enzymes extracted from Ascomycete fungi are extracted from at least one of A. niger, P. chrysogenum, T harzianum and P. citrinum.

10. The method according to claim 9, wherein the biomass from step b) is incubated with a mixture of enzymes extracted from A. niger, P. chrysogenum, T harzianum and P. citrinum.

11. The method according to claim 1, wherein each incubation is for less than 24 hours.

12. The method according to claim 1, wherein the biomass is comprised of biomass by-products produced by wineries, cereal crop farms, sugar cane plantations or forest productions.

13. The method according to claim 12, wherein the biomass is comprised of biomass by-products produced by wineries.
